(19) United States
(12) Patent Application Publication
(10) Pub. No.: US 2016/0067311 A1
(43) Pub. Date: Mar. 10, 2016

(54) USE OF ELSIGLUTIDE TO TREAT GASTROINTESTINAL MUCOSITIS INCLUDING CHEMOTHERAPY-INDUCED DIARRHEA

(71) Applicant: Helsinn Healthcare SA, Lugano/Pazzallo (CH)

(72) Inventors: Ruben GIORGINO, Allschwil (CH); Simona RONCORONI, Villa Guardia (como) (IT); Selma CALCAGNILE, Appiano Gentile (como) (IT); Fabio TRENTO, Colverde (IT); Riccardo SPEZIA, Milano (IT); Cecilia MORESINO, Morbio Inferiore (CH)

(73) Assignee: HELSINN HEALTHCARE SA, Lugano/Pazzallo (CH)

(21) Appl. No.: 14/842,250

(22) Filed: Sep. 1, 2015

Related U.S. Application Data
(60) Provisional application No. 62/048,520, filed on Sep. 10, 2014.

(51) Int. Cl. A61K 38/26

(52) U.S. Cl. CPC A61K 38/26

(57) ABSTRACT

The invention relates to the use of elsiglutide to prevent or reduce the occurrence of gastrointestinal damage caused by chemotherapeutic agents, including gastrointestinal mucositis and chemotherapy-induced diarrhea (CID).

### FIELD OF THE INVENTION

The invention relates to the use of elsiglutide to protect against and prevent gastrointestinal organ-specific toxicities induced by chemotherapeutic agents. In particular, this invention relates to the use of elsiglutide to prevent and reduce the occurrence or severity of gastrointestinal mucositis, and its clinical manifestations including chemotherapy-induced diarrhea.

### BACKGROUND OF THE INVENTION

Gastrointestinal (GI) damage and dysfunction are well-known side effects of cancer-chemotherapy treatments and can be debilitating and potentially life threatening (Richardson and Dobish, J Oncol Pharm Practice 2007; 13:181-198; Grem et al., J Clin Oncol 1987; 5(10):1704; Petrelli et al., J Clin Oncol 1989; 7(10):1419-1426). Chemotherapy administration is frequently associated with mucositis, diarrhea (chemotherapy-induced diarrhea (CID), bacterial translocation, malabsorption, abdominal cramping, gastrointestinal bleeding and vomiting. These side effects are clinical consequences of the structural and functional damage of the intestinal epithelium and frequently make it necessary to decrease the dose and frequency of chemotherapy, thus negatively affecting the overall clinical outcome of the patients. The intestinal mucositis and diarrhea can result in severe dehydration, electrolyte imbalances, sepsis due to bacterial translocation, cardiovascular instability, and renal insufficiency. Each of these factors contribute to a high morbidity and mortality associated with CID (Rubenstein et al., Cancer 2004; 100(9 Suppl):2026-2046; Rapoport et al., J Clin Oncol 1999; 17(8):2446-2453; Petrelli et al., J Clin Oncol 1987; 5(10):1559-1565).

The stem cells of the small intestinal mucosa are particularly susceptible to the cytotoxic effects of chemotherapy due to their rapid rate of proliferation (Keefe et al., Gut 2000; 47: 632-7). Chemotherapy-induced damage to the small intestinal mucosa is referred to as gastrointestinal mucositis and is characterized by absorptive and barrier impairments of the small intestine. For example, it has been shown that the broadly used chemotherapeutic agents 5-fluorouracil (5-FU), irinotecan and methotrexate increase apoptosis leading to villus atrophy and crypt hypoplasia in the small intestine of rodents (Keefe et al., Gut 47: 632-7, 2000; Gibson et al., J Gastroenterol Hepatol. September; 18(9):1095-1100, 2003; Tamaki et al., J Int Med Res. 31(1):6-16, 2003). Chemotherapeutic agents have been shown to increase apoptosis in intestinal crypts at 24 hours after administration and subsequently to decrease villus area, crypt length, mitotic count per crypt, and enterocyte height three days after chemotherapy in humans (Keefe et al., Gut 2000; 47: 632-7). Thus, structural changes within the small intestine can lead directly to intestinal dysfunction and in some cases diarrhea.

Gastrointestinal mucositis after cancer chemotherapy is an increasing problem that is essentially untreatable once established, although it gradually remits. Studies conducted with the commonly used cytostatic cancer drugs 5-FU and irinotecan have demonstrated that efficacious doses of these drugs predominantly affects structural integrity and function of the small intestine while the colon is less sensitive and mainly responds with increased mucus formation (Gibson et al., J Gastroenterol Hepatol. September; 18(9):1095-1100, 2003; Tamaki et al., J Int Med Res. 31(1):6-16, 2003).

Reported data about the frequency of CID and its severity can vary significantly and have been derived mostly from clinical trials on colorectal cancer treatments. Data from the late 1990s demonstrated CID incidence to vary from a general 10-20% to the 50-80% found in colorectal cancer patients treated with 5-fluorouracil (5-FU) or irinotecan (with up to 30% of these patients experiencing severe diarrhea, a life-threatening condition that may lead to hospitalization, and death in 2-5% of cases) (see Stein et al., Ther Adv Med Oncol 2010, 2: 51-63; Gibson and Stringer, Curr Opin Support Palliat Care 2009, 3: 31-35; Benson et al., J Clin Oncol 2004, 22: 2918-26; Conti et al., J Clin Oncol 1996, 14: 709-15; Leichman et al., J Clin Oncol 1995, 13: 1303-1311; Rothenberg et al., Cancer 1999, 85: 786-95). Two retrospective studies have also been conducted that confirmed highest frequencies of diarrhea from chemotherapy regimens based on 5-FU, irinotecan, capecitabine and/or oxaliplatin (Arbuckle et al., The Oncologist 2000, 5: 250-9; Goldberg et al., J Support Oncol 2005, 3: 227-32). Importantly, some authors argue that CID events may be underreported as well as under-recognized and underestimated in the clinical setting (Arbuckle et al., The Oncologist 2000, 5: 250-9; Cirillo et al., Annals of Oncology 2009, 20: 1929-35). This seems to be confirmed by the large variability of frequencies reported in literature.

Generally, CID is a dose-related adverse effect engendered by a multi-factorial process, through which the acute cytotoxic damage to the intestinal mucosa (including loss of intestinal epithelium, superficial necrosis and inflammation of the bowel wall) causes an imbalance between absorption and secretion in the small intestine (Stein et al., Ther Adv Med Oncol 2010, 2: 51-63; Gibson and Stringer, Curr Opin Support Palliat Care 2009, 3: 31-35; Keefe, Curr Opin Oncol 2007, 19: 323-27). Except for irinotecan-induced early onset diarrhea, a dose-dependent adverse reaction occurring within 24 hours after the drug administration, the onset of CID episodes generally occurs a few days following administration, at all dose levels. For irinotecan, the median time to late-CID onset is reported between 6 to 11 days following administration (Stein et al., Ther Adv Med Oncol 2010, 2: 51-63). Besides standard cytotoxic agents, recent findings also highlight that biological targeted chemotherapeutic agents (such as tyrosine-kinase inhibitors) frequently cause diarrhea, which may occur in up to 60% of patients treated with certain drugs, such as lapatinib (Stein et al., Ther Adv Med Oncol 2010, 2: 51-63; Lowell, 2012 MASCC/ISOO International Symposium on Supportive Cancer Care). Therefore, in the future, more patients with non-gastrointestinal cancers might also experience treatment-induced diarrhea.

The prompt assessment, diagnosis and implementation of appropriate management strategies are essential for the prevention of potentially severe and also fatal consequences of CID (Benson et al., J Clin Oncol 2004, 22: 2918-26; Maroun et al., Curr Oncol 2007, 14: 13-20; Common Terminology Criteria for Adverse Events (CTCAE) Version 4.0 Published: May 28, 2009 (v4.03: Jun. 14, 2010): http://evs.nci.nih.gov/ftp1/CTCAE/CTCAE_{—}4.03_{—}2010-06-14_QuickReference_{—}8.5×11.pdf). In literature, the clinical assessment of diarrhea severity is most commonly measured by the NCI-CTC criteria, which define 5 severity degrees of diarrhea, based on the number of bowel movements experienced per day above baseline (Common Terminology Criteria for Adverse Events (CTCAE) Version 4.0 Published: May 28, 2009 (v4.03: Jun. 14, 2010): http://evs.nci.nih.gov/ftp1/CTCAE/CTCAE_{—}4.03_{—}2010-06-14_QuickReference_{—}8.5×11.pdf). Although the NCI-CTC criteria do not evaluate stool volume and consistency, or diarrhea duration, this scale is widely accepted and used in clinical practice and clinical research (Saltz L B, Understanding and managing chemotherapy-induced diarrhea. J Support Oncol 2003, 1:35-46). Furthermore, several studies have accentuated the psychosocial consequences of CID and suggested the importance of evaluating the patient's perception and quality of life in order to achieve a comprehensive diagnostic assessment (Chen et al., Cancer 2010, 116: 1879-86; Flores et al., Gastrointest Cancer Res 2012, 5:119-24).

Current pharmacologic treatments for manifestations of gastrointestinal mucositis such as CID are palliative at best; they help to control and treat symptoms rather than to prevent the onset of diarrhea. Non-analgesic opiates such as loperamide are commonly employed because of their ability to promote intestinal fluid re-absorption and to slow down intestinal motility. Loperamide is commonly used as a standard first-line therapy for Grades 1-2 diarrhea. Octreotide, a synthetic somatostatin analogue has been shown to reduce the secretion of some intestinal hormones and promote intestinal absorption of fluids and electrolytes. While it is not approved for this use, and has inadequate evidence demonstrating its utility, octreotide is often used for second line treatment of loperamide refractory CID or for grades 3-4 diarrhea. Parenteral hydration and electrolyte supplementation and in severe cases, total parenteral nutrition are used as well. See, e.g., Benson et al., J Clin Oncol 2004; 22(14):2918-2926. Other therapeutic options are available and under study, such as probiotics, but are not currently recommended for practice (Benson et al., J Clin Oncol 2004; 22(14):2918-2926; Muehlbauer et al., Clin J Oncol Nurs 2009, 13: 336-41).

Because the currently available therapies for CID only offer symptomatic relief, there is a great medical need for agents addressing the root causes of the disease that prevent or decrease the intestinal mucosal damage caused by chemotherapy.

Glucagon-like-peptide-2 (GLP-2) is a 33-amino-acid peptide released from the post-translational processing of proglucagon in the enteroendocrine L cells of the intestine and in specific regions of the brainstem. It is co-secreted together with glucagon-like peptide 1 (GLP-1), oxyntomodulin and glicentin, in response to nutrient ingestion. GLP-2 induces significant growth of the small intestinal mucosal epithelium via the stimulation of stem cell proliferation in the crypts and inhibition of apoptosis on the villi (Drucker et al. Proc Natl Acad Sci USA. 1996, 93:7911-6). GLP-2 also inhibits gastric emptying and gastric acid secretion (Wojdemann et al. J Clin Endocrinol Metab. 1999, 84:2513-7), enhances intestinal barrier function (Benjamin et al. Gut. 2000, 47:112-9.), stimulates intestinal hexose transport via the upregulation of glucose transporters (Cheeseman, Am J Physiol. 1997, R1965-71), and increases intestinal blood flow (Guan et al. Gastroenterology. 2003, 125, 136-47).

The demonstrated specific and beneficial effects of GLP-2 in the small intestine has raised much interest as to the use of GLP-2 in the treatment of intestinal disease or injury (Sinclair and Drucker, Physiology 2005: 357-65). Furthermore GLP-2 has been shown to prevent or reduce mucosal epithelial damage in a wide number of preclinical models of gut injury, including chemotherapy-induced mucositis, ischemia-reperfusion injury, dextran sulfate-induced colitis and genetic models of inflammatory bowel disease (Sinclair and Drucker, Physiology 2005:357-65).

GLP-2 is secreted as a 33 amino acid peptide having the sequence HADGSFSDEMNTILDNLAARDFINWLIQTKITD (SEQ ID NO: 2). It is rapidly cleaved at the Alanine (A) in position 2 of the N-terminus to the inactive human GLP-2 (3-33) by the enzyme dipeptidyl peptidase-4 (DPP IV). This rapid enzymatic degradation of GLP-2(1-33), in addition to renal clearance results in a half-life of about 7 minutes for the peptide (Tavares et al., Am. J. Physiol. Endocrinol. Metab. 278:E134-E139, 2000).

U.S. Pat. Nos. 7,745,403 and 7,563,770 disclose GLP-2 analogues which comprise one of more substitutions as compared to wild-type GLP-2. One of the described GLP-2 analogues is ZP1846 (elsiglutide). A comparison of the sequences of GLP-2 and elsiglutide is provided below:

elsiglutide:
(SEQ ID NO: 1)
HGEGSFSSELSTILDALAARDFIAWLIATKITDKKKKKK
GLP-2:
(SEQ ID NO: 2)
HADGSFSDEMNTILDNLAARDFINWLIQTKITD.

U.S. Pat. Nos. 7,745,403 and 7,563,770 propose the use of GLP-2 analogues, including elsiglutide, for preventing or ameliorating side effects of chemotherapy, including chemotherapy-induced diarrhea (CID). GLP-2 analogues appear to act in CID by inhibiting enterocyte and crypt cell apoptosis and increasing crypt cell proliferation, thus providing new cells to replace the damaged intestinal epithelium following chemotherapy.

A planned experimental trial of elsiglutide was reported on clinicaltrials.gov sometime around Feb. 21, 2012. The official title of the trial was Phase II, Double-blind, Randomized, Two-stage, Placebo-controlled Proof of Concept Study in Colorectal Cancer Patients Receiving 5-FU Based Chemotherapy to Assess the Efficacy of Elsiglutide (ZP1846) Administered s.c. in the Prevention of Chemotherapy Induced Diarrhea (CID). Clinicaltrials.gov reports the following brief summary of the study: The main objective of this study will be to obtain data on the efficacy of elsiglutide in preventing Chemotherapy Induced Diarrhea (CID) in patients with colorectal cancer receiving 5-FU based chemotherapy (FOLFOX4 or FOLFIRI regimen) in comparison to placebo. The results of the study are not reported in clinicaltrials.gov, but are reported for the first time herein.

### SUMMARY OF THE INVENTION

As specified in the Background Section, there is a great need in the art to prevent/ameliorate toxicities in the gastrointestinal tract associated with anti-cancer chemotherapies, particularly the CID resulting from gastrointestinal mucositis. The present invention addresses these and other needs by providing the methods and compositions described below.

The present invention is based on the discovery that the occurrence of chemotherapy-induced diarrhea (CID) is prevented or its severity is reduced upon administration of the GLP-2 analog elsiglutide. In particular the invention is based on the unexpected finding that the administration of elsiglutide provides a protective effect against CID that extends long after the elsiglutide is administered. The effect is especially pronounced for diarrhea of Grade≧2 as determined by National Cancer Institute Common Toxicity Criteria for Diarrhea (CTCAE v.4.03). In one human clinical study, 24 mg of elsiglutide was administered subcutaneously (s.c.) on days 1, 2, 3 and 4 from the beginning of a 14 day chemotherapy cycle. The chemotherapy was administered on days 1 and 2 of the cycle. Even though the elsiglutide was administered for only a few days at the beginning of the chemotherapy cycle, the elsiglutide was able to lower the incidence of Grade 2 and less diarrhea events during the entire period of the chemotherapy cycle, including on days 5 through 6 when the occurrence of diarrhea is shown to be the greatest, and levels of citrulline (a marker of intestinal damage) are decreased compared to baseline values.

Therefore, in a first embodiment the invention provides a method for preventing or reducing the occurrence of grade 2 or higher diarrhea resulting from an anti-cancer chemotherapy in a subject in need thereof, which method comprises administering to the subject a therapeutically effective amount of elsiglutide in an elsiglutide regimen, wherein the elsiglutide regimen preferably comprises daily administration of elsiglutide for four consecutive days, preferably commencing at the start of the chemotherapy cycle.

More generally speaking, the invention provides a method for treating gastrointestinal mucositis, or otherwise preventing or reducing gastrointestinal (GI) damage and/or dysfunction associated with an anti-cancer chemotherapy in a subject in need thereof, which method comprises administering to the subject a therapeutically effective amount of elsiglutide in an elsiglutide regimen, wherein the elsiglutide regimen preferably comprises daily administration of elsiglutide for a plurality of consecutive days, preferably commencing at the start of the chemotherapy cycle and ending prior to the conclusion of the chemotherapy cycle.

Another embodiment relates to the use of elsiglutide to prevent or reducing the occurrence of or severity of gastrointestinal mucositis (including CID) in cancer patients receiving antibody therapy for their cancer, with or without treatment with a small cytotoxic agent. This embodiment is particularly useful in preventing or reducing the occurrence of grade 2 or higher CID. Thus, in still another embodiment the invention provides a method for preventing or reducing gastrointestinal mucositis in a cancer patient receiving antibody therapy, including grade 2 or higher CID, by administering to the subject a therapeutically effective amount of elsiglutide in an elsiglutide regimen, wherein the elsiglutide regimen preferably comprises daily administration of elsiglutide for four consecutive days, preferably commencing at the start of the antibody cycle.

Still another embodiment relates to the use of elsiglutide to prevent GI damage as detected by means of certain citrulline levels during chemotherapy. In this embodiment, the invention provides a method for preventing GI damage by maintaining citrulline levels in a subject receiving chemotherapy, comprising administering to said subject a therapeutically effective amount of elsiglutide.

Additional embodiments and advantages of the invention will be set forth in part in the description which follows, and in part will be obvious from the description, or may be learned by practice of the invention. The embodiments and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 shows the proportion of patients with diarrhea of any grade, by day on days 1-14, with or without elsiglutide administration in study TIDE-11-10.

FIG. 2 shows the proportion of patients with diarrhea of grade≧2, by day on days 1-14, with or without elsiglutide administration in study TIDE-11-10.

### DETAILED DESCRIPTION OF THE INVENTION

### Definitions

When a peptide active ingredient is referred to herein in its native form, it will be understood to include all pharmaceutically acceptable salts thereof. Thus, references to elsiglutide include elsiglutide hydrochloride, and other pharmaceutically acceptable salts of elsiglutide.

As used herein, the terms “elsiglutide” and “ZP1846” are used interchangeably to refer to a GLP-2 peptide analog having amino acid sequence:

(SEQ ID NO: 1)
HGEGSFSSELSTILDALAARDFIAWLIATKITDKKKKKK

The terms also encompass peptides provided in the form of a salt. Salts include pharmaceutically acceptable salts such as, e.g., acid addition salts and basic salts. Non-limiting examples of acid addition salts include hydrochloride salts, citrate salts and acetate salts. Non-limiting examples of basic salts include salts where the cation is selected from alkali metals, such as sodium and potassium, alkaline earth metals, such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl. Other examples of pharmaceutically acceptable salts are described in “Remington's Pharmaceutical Sciences”, 17th edition. Ed. Alfonso R. Gennaro (Ed.), Mark Publishing Company, Easton, Pa., U.S.A., 1985 and more recent editions, and in the Encyclopaedia of Pharmaceutical Technology.

The terms “cancer chemotherapy” and “chemotherapy” are used interchangeably herein to refer to a therapy of cancer by administering an anti-cancer agent. The terms “anti-cancer agent” and “chemotherapeutic agent” are used herein to refer to any chemical compound which is used to treat cancer. Chemotherapeutic agents are well known in the art (see, e.g., Gilman A. G., et al., The Pharmacological Basis of Therapeutics, 8th Ed., Sec 12:1202-1263 (1990)). Specific non-limiting examples of chemotherapeutic agents are provided throughout the specification and include, for example, FOLFOX (a chemotherapy regimen for treatment of colorectal cancer, which comprises administration of folinic acid (leucovorin), fluorouracil (5-FU), and oxaliplatin) and FOLFIRI (a chemotherapy regimen for treatment of colorectal cancer, which comprises administration of folinic acid (leucovorin), fluorouracil (5-FU), and irinotecan), as well as administration of targeted monoclonal antibody therapy (e.g., bevacizumab, cetuximab, or panitumumab) alone or in combination with chemotherapeutic agents.

The term “chemotherapy cycle” is used herein to refer to a period of time between the initial administration of an anti-cancer agent and its repeat administration. For example, the cycle of the FOLFOX4 chemotherapy discussed in the Examples section below includes 14 days, wherein anti-cancer agents are administered only for the first 2 days of the cycle as follows: Day 1: oxaliplatin 85 mg/m² IV infusion and leucovorin 200 mg/m² IV infusion both given over 120 minutes at the same time in separate bags, followed by 5-FU 400 mg/m² IV bolus given over 2-4 minutes, followed by 5-FU 600 mg/m² IV infusion as a 22-hour continuous infusion; Day 2: leucovorin 200 mg/m² IV infusion, followed by 5-FU 400 mg/m² IV bolus given over 2-4 minutes, followed by 5-FU 600 mg/m² IV infusion as a 22-hour continuous infusion. Similarly, the cycle of the FOLFIRI chemotherapy discussed in the Examples section, below, includes 14 days, wherein anti-cancer agents are administered only for the first 2 days of the cycle as follows: irinotecan (180 mg/m² IV over 90 minutes) concurrently with folinic acid (400 mg/m² [or 2×250 mg/m²] IV over 120 minutes), followed by fluorouracil (400-500 mg/m² IV bolus) then fluorouracil (2400-3000 mg/m² intravenous infusion over 46 hours). Bevacizumab is usually given intravenously every 14 days, although the frequency can be dose dependent (for example 5 mg/kg by intravenous infusion every two weeks or 7.5 mg/kg by intravenous infusion every three weeks). In colon cancer, it is given in combination with the chemotherapy drug 5-FU (5-fluorouracil), leucovorin, and oxaliplatin or irinotecan. One recommended dose and schedule for cetuximab is 400 mg/m² administered intravenously as a 120-minute infusion as an initial dose, followed by 250 mg/m² infused over 30 minutes weekly, preferably in combination with FOLFIRI.

The terms “co-administered” and “co-administration” broadly refer to administration of two or more components, compounds or compositions (e.g., a chemotherapeutic agent and elsiglutide), wherein said components, compounds or compositions can be administered either simultaneously (in one composition or in two or more separate compositions) or sequentially.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Where particular values are described in the application and claims, unless otherwise stated, the term “about” is implicit and in this context means within an acceptable error range for the particular value.

In the context of the present invention insofar as it relates to any of the disease conditions recited herein, the terms “prevent,” “prevention,” “treat,” “treatment” and the like are synonymous and mean to reduce the occurrence of a symptom or condition, or to relieve or alleviate at least one symptom associated with such condition, or to slow or reverse the progression of such condition. Within the meaning of the present invention, the terms also denotes to arrest, delay the onset (i.e., the period prior to clinical manifestation of a disease) and/or reduce the risk of developing or worsening a disease. For example, in connection with cancer the term “prevent,” “treat” or “reduce the occurrence of,” may mean that a therapy has been demonstrated in a prospectively designed clinical trial to reduce the incidence or occurrence of a clinical endpoint, symptom or condition such as diarrhea or gastrointestinal mucositis.

As used herein the term “therapeutically effective” applied to dose or amount refers to that quantity of a compound or pharmaceutical composition that is sufficient to result in a desired activity upon administration to a subject in need thereof. Within the context of the present invention, when the term “therapeutically effective” is used in connection with elsiglutide, it refers to an amount of elsiglutide or a pharmaceutical composition containing elsiglutide that is effective to prevent side effects or reduce the incidence, occurrence or severity of side effects of cancer chemotherapy such as damage to the gastrointestinal mucosa or diarrhea. Note that when a combination of active ingredients is administered (e.g., a combination of elsiglutide and another compound effective for ameliorating or preventing side effects of cancer chemotherapy) the effective amount of the combination may or may not include amounts of each ingredient that would have been effective if administered individually.

The phrase “pharmaceutically acceptable”, as used in connection with compositions of the invention, refers to molecular entities and other ingredients of such compositions that are physiologically tolerable and do not typically produce untoward reactions when administered to a subject (e.g., a mammal such as a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “subject” refers to any mammal. In a preferred embodiment, the subject is human.

As used in this specification and the appended claims, the singular forms “a,” “an,” and “the” include plural references unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

When a range of values can be used to describe a particular regimen, it will be understood that the range can be defined by selectively combining any one of the lower end of variables described in the specification with any one of the upper end of variables described in the specification that is mathematically possible.

Throughout this application, whenever a standard is given with reference to a test or methodology currently accepted and applied in the scientific community, the standard will be understood to be evaluated with respect to the test or methodology as it is reported in the published literature on Jul. 1, 2014.

### Discussion

In a first embodiment the invention provides a method for preventing or reducing the occurrence of grade 2 or higher diarrhea as determined by National Cancer Institute Common Toxicity Criteria for Diarrhea (CTCAE v.4.03) resulting from an anti-cancer chemotherapy cycle in a subject in need thereof, which method comprises administering to the subject a therapeutically effective amount of elsiglutide in an elsiglutide regimen, wherein the elsiglutide regimen preferably comprises daily administration of elsiglutide, preferably for four consecutive days commencing at the start of the chemotherapy cycle.

More generally speaking, the invention provides a method for preventing or reducing gastrointestinal (GI) damage and/or dysfunction associated with an anti-cancer chemotherapy cycle in a subject in need thereof, which method comprises administering to the subject a therapeutically effective amount of elsiglutide in an elsiglutide regimen, wherein the elsiglutide regimen preferably comprises daily administration of elsiglutide for a plurality of consecutive days, preferably commencing at the start of the chemotherapy cycle and ending prior to the conclusion of the chemotherapy cycle.

The invention can also be used to prevent and reduce gastrointestinal mucositis induced by antibody chemotherapy, and in this embodiment the invention provides a method for preventing gastrointestinal mucositis in a cancer patient receiving antibody therapy alone or in combination with one or more chemotherapy agents, including grade 2 or higher CID, by administering to the subject a therapeutically effective amount of elsiglutide in an elsiglutide regimen, wherein the elsiglutide regimen preferably comprises daily administration of elsiglutide, preferably for four consecutive days commencing at the start of the antibody cycle.

Still another embodiment relates to the use of elsiglutide to prevent GI damage as detected by means of certain citrulline levels during chemotherapy. In this embodiment, the invention provides a method for preventing GI damage by maintaining citrulline levels in a subject receiving chemotherapy, comprising administering to said subject a therapeutically effective amount of elsiglutide. By “maintaining,” it is meant that the elsiglutide preferably prevents the citrulline levels of citrulline from dropping 20% or more, 40% or more, 60% or more, or even 80% or more, from the level that citrulline would drop in the absence of such elsiglutide.

Elsiglutide and a chemotherapeutic agent(s)are preferably administered concurrently for two or more days, with the elsiglutide administration beginning on the same day that the chemotherapy cycle begins, although it is feasible to administer or at least initiate the elsiglutide administration before the administration of the chemotherapeutic agent(s) begins, or to administer elsiglutide after the administration of the chemotherapeutic agent(s) concludes (i.e., during the days of the chemotherapy cycle when the chemotherapeutic agent(s) is no longer administered). When the chemotherapy comprises multiple cycles, such as 2, 3, 4 or more cycles, elsiglutide is preferably administered during each of the cycles. When administered on a daily basis, elsiglutide can be administered one or more times during the day, but it is preferably only administered once daily.

The elsiglutide regimen preferably comprises elsiglutide administration daily for 1, 2, 3, 4, 5, or six days of the chemotherapy cycle, or anywhere between these time periods (such as 1-5 days), although 4 days appears to be adequate. The regimen is also preferably initiated at the start of the chemotherapy cycle, although the regimen can also be initiated as many as 1, 2, 3, 4 or 5 days prior to the initiation of the chemotherapy cycle. The regimen is also preferably performed on consecutive days, although dosing for non-consecutive daily periods can also be envisioned.

A chemotherapy cycle may comprise administration of chemotherapy for 1 or more, 3 or more, 5 or more, 7 or more, 9 or more, or even 10 or more consecutive days during the cycle, or anywhere between these time periods (such as 1 to up to 5 days). The chemotherapy cycle might last for one week, two weeks, three weeks, four weeks, or even more, or anywhere in between these time periods. It has been found herein that a limited period of elsiglutide administration is effective to prevent or reduce the occurrence or severity of gastrointestinal mucositis or CID throughout a 14 day chemotherapy cycle, including on days 5 through 9 when the incidence of mucositis or CID is shown to be most pronounced.

In a particularly preferred embodiment, the methods are used to prevent and reduce the occurrence or severity of more severe cases of CID, grade 2 or higher as determined by the National Cancer Institute Common Toxicity Criteria for Diarrhea (CTCAE v.4.03).

According to the present invention, elsiglutide can be used to reduce toxicity of a wide range of different chemotherapeutic agents, prodrugs of such chemotherapeutic agents, and chemotherapy regimens. While some chemotherapy agents and regimens are better known for producing CID and damaging the gastrointestinal mucosa, the invention can also be practiced to reduce subclinical occurrences of CID or damage to the gastrointestinal mucosa from practically any chemotherapy agent. Non-limiting examples of such agents include anti-metabolites such as pyrimidine analogs (e.g., 5-fluorouracil [5-FU], floxuridine, capecitabine, gemcitabine and cytarabine) and purine analogs, folate antagonists and related inhibitors (e.g., mercaptopurine, thioguanine, pentostatin and 2-chlorodeoxyadenosine (cladribine)); antiproliferative/antimitotic agents including natural products such as vinca alkaloids (e.g., vinblastine, vincristine, and vinorelbine), microtubule disruptors such as taxanes (e.g., paclitaxel, docetaxel), vincristin, vinblastin, nocodazole, epothilones and navelbine, epidipodophyllotoxins (e.g., etoposide, teniposide), DNA damaging agents (e.g., actinomycin, amsacrine, anthracyclines, bleomycin, busulfan, camptothecin, carboplatin, chlorambucil, cisplatin, nedaplatin, cyclophosphamide, cytoxan, dactinomycin, daunorubicin, doxorubicin, epirubicin, aclarubicin, purarubicin, hexamethyhnelamineoxaliplatin, iphosphamide, melphalan, merchlorehtamine, mitomycin, mitoxantrone, nitrosourea, nimustine, ranimustine, estramustine, plicamycin, procarbazine, taxol, taxotere, teniposide, triethylenethiophosphoramide and etoposide (VP16)); antibiotics (e.g., dactinomycin (actinomycin D), daunorubicin, doxorubicin (adriamycin), idarubicin, anthracyclines, mitoxantrone, bleomycins, plicamycin (mithramycin), pleomycin, peplomycin, mitomycins (e.g., mitomycin C), actinomycins (e.g., actinomycin D), zinostatinstimalamer); enzymes (e.g., L-asparaginase); neocarzinostatin; antiplatelet agents; antiproliferative/antimitotic alkylating agents such as nitrogen mustards (e.g., mechlorethamine, cyclophosphamide and analogs, imidazol carboxamide, melphalan, chlorambucil, nitrogen mustard-N-oxide hydrochloride, ifosfamide), ethylenimines and methylmelamines (e.g., hexamethylmelamine, thiotepa, carboquone, triethylene thiophospharamide), alkyl sulfonates (e.g., busulfan, isoprosulfan tosylate), nitrosoureas (e.g., carmustine (BCNU) and analogs, streptozocin), trazenes-dacarbazinine (DTIC); epoxide type compounds (e.g., mitobronitol); antiproliferative/antimitotic antimetabolites such as folic acid analogs (e.g., methotrexate); platinum coordination complexes (e.g., cisplatin, carboplatin, oxaliplatin), procarbazine, hydroxyurea, mitotane, aminoglutethimide; hormones, hormone analogs (e.g., estrogen, tamoxifen, goserelin, bicalutamide, nilutamide) and aromatase inhibitors (e.g., letrozole, anastrozole); anticoagulants (e.g., heparin, synthetic heparin salts and other inhibitors of thrombin); fibrinolytic agents (e.g., tissue plasminogen activator, streptokinase and urokinase), aspirin, dipyridamole, ticlopidine, clopidogrel, abciximab; antimigratory agents; antisecretory agents (e.g., breveldin); immunosuppressives (e.g., cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blockers; nitric oxide donors; antisense oligonucleotides; antibodies (e.g., trastuzumab); cell cycle inhibitors and differentiation inducers (e.g., tretinoin); mTOR inhibitors, topoisomerase inhibitors (e.g., doxorubicin (adriamycin), amsacrine, camptothecin, daunorubicin, dactinomycin, eniposide, epirubicin, etoposide, idarubicin, mitoxantrone, topotecan, irinotecan); growth factor signal transduction kinase inhibitors (erlotinib, sorafenib, lapatinib); mitochondrial dysfunction inducers; chromatin disruptors; sobuzoxane; tretinoin; pentostatin; flutamide; porphimer natrium; fadrozole; procarbazine; aceglatone, and mitoxantrone.

Non-limiting examples of gastrointestinal (GI) damage and/or dysfunction associated with anti-cancer chemotherapies include, for example, chemotherapy-induced diarrhea (CID), nausea, vomiting, anorexia, body weight loss, heavy feeling of stomach, constipation, stomatitis, and esophagitis.

The methods of the invention can be used in subjects suffering from a broad range of cancers for which treatment could induce CID or gastrointestinal damage. Non-limiting examples of relevant cancers include, e.g., breast cancer, prostate cancer, multiple myeloma, transitional cell carcinoma, lung cancer (e.g., non-small cell lung cancer (NSCLC)), renal cancer, thyroid cancer and other cancers causing hyperparathyroidism, adenocarcinoma, leukemia (e.g., chronic myeloid leukemia, acute myeloid leukemia, chronic lymphocytic leukemia, acute lymphocytic leukemia), lymphoma (e.g., B cell lymphoma, T cell lymphoma, non-Hodgkins lymphoma, Hodgkins lymphoma), head and neck cancer, esophageal cancer, stomach cancer, colon cancer, intestinal cancer, colorectal cancer, rectal cancer, pancreatic cancer, liver cancer, cancer of the bile duct, cancer of the gall bladder, ovarian cancer, uterine endometrial cancer, vaginal cancer, cervical cancer, bladder cancer, neuroblastoma, sarcoma, osteosarcoma, malignant melanoma, squamous cell cancer, bone cancer, including both primary bone cancers (e.g., osteosarcoma, chondrosarcoma, Ewing's sarcoma, fibrosarcoma, malignant fibrous histiocytoma, adamantinoma, giant cell tumor, and chordoma) and secondary (metastatic) bone cancers, soft tissue sarcoma, basal cell carcinoma, angiosarcoma, hemangiosarcoma, myxosarcoma, liposarcoma, osteogenic sarcoma, angiosarcoma, endotheliosarcoma, lymphangiosarcoma, lymphangioendotheliosarcoma, synovioma, testicular cancer, uterine cancer, gastrointestinal cancer, mesothelioma, leiomyosarcoma, rhabdomyosarcoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, choriocarcinoma, seminoma, embryonal carcinoma, Wilms' tumor, epithelial carcinoma, glioma, glioblastoma, astrocytoma, medulloblastoma, craniopharyngioma, ependymoma, pinealoma, hemangioblastoma, acoustic neuroma, oligodendroglioma, meningioma, retinoblastoma, medullary carcinoma, thymoma, sarcoma, etc.

### Methods for Administering Elsiglutide and Elsiglutide Compositions

Specific elsiglutide doses useful in the methods of the invention will depend on the type of chemotherapy side effects to be treated, the severity and course of these side effects, previous therapy, the patient's clinical history and response to chemotherapy and elsiglutide, as well as the discretion of the attending physician. In one specific embodiment, such doses range from 5 to 80 or from 10 to 40 mg/ day.

The administration of elsiglutide according to the methods of the invention can be performed by any suitable route. Specific non-limiting examples of useful routes of administration include subcutaneous, intravenous (IV), intraperitoneal (IP), and intramuscular.

In certain embodiments, elsiglutide is formulated in a pharmaceutical composition with a pharmaceutically acceptable carrier or excipient. In certain embodiments, elsiglutide is combined in a pharmaceutical composition together with another compound effective for ameliorating or preventing side effects of cancer chemotherapy. The formulations used in the methods of the invention may conveniently be presented in unit dosage form and may be prepared by methods known in the art. The amount of active ingredients that can be combined with a carrier material to produce a single dosage form will vary depending upon the host being treated and the particular mode of administration. The amount of active ingredients that can be combined with a carrier material to produce a single dosage form will generally be that amount of the compound which produces a therapeutic effect.

In general, the formulations can be prepared with a liquid carrier, or a finely divided solid carrier, or both, and then, if necessary, shaping the product. Pharmaceutical compositions suitable for parenteral administration may comprise elsiglutide in combination with one or more pharmaceutically acceptable sterile isotonic aqueous or nonaqueous solutions, dispersions, suspensions or emulsions, or sterile powders which may be reconstituted into sterile injectable solutions or dispersions just prior to use.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Initial Clinical Characterization of Elsiglutide

1.1. Summary of Clinical Data

Data on human exposure to elsiglutide generated so far originate from three clinical trials, one performed in healthy subjects (study 06-013) and two in cancer patients (TIDE-09-04, TIDE-11-10). The main objective of the first two studies was the evaluation of the safety, tolerability and maximum tolerated dose (MTD) of ascending elsiglutide doses administered as s.c. (both studies) and i.v. (only study 06-013) bolus injections. In study 06-013 elsiglutide was administered as single i.v. or s.c. bolus, while in study TIDE-09-04 each dose was administered as s.c. bolus on 4 consecutive days. Study TIDE-11-10 was a phase II proof of concept study that mainly evaluated the efficacy of elsiglutide administered as 24 mg daily s.c. bolus injections for 4 consecutive days in preventing CID in patients with colorectal cancer receiving 5-FU based chemotherapy (FOLFOX4 or FOLFIRI regimen). The secondary objectives of all three studies included the evaluation of the pharmacokinetics of elsiglutide and its major metabolites in humans.

### Pharmacokinetics

Elsiglutide has a short half-life (0.4 h) and is quickly eliminated from blood circulation after single i.v. administration. Data, although limited to 1 subject only, indicate an absolute bioavailability of 0.29%. The two human metabolites of elsiglutide, ZP2242 and ZP2712, have longer half-lives (6-9 h) with overall plasma levels substantially higher than elsiglutide. ZP2242 is the major metabolite and revealed an AUC at least 10 times higher than that of ZP2712. In general, exposure to the metabolite ZP2242 was >25-fold higher than exposure to the parent ZP1846 (elsiglutide) on Day 1. Exposure to the metabolite ZP2712 was >4 fold higher than exposure to the parent ZP1846 (elsiglutide) on Day 1.

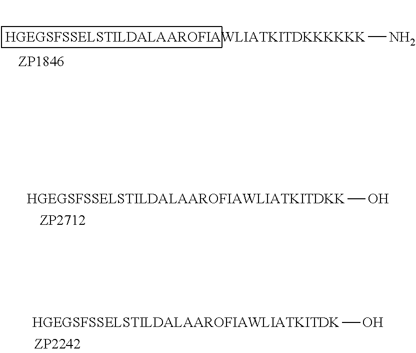

In study TIDE-09-04 the pharmacokinetics of elsiglutide and the metabolites ZP2242 and ZP2712 appeared to be dose independent, i.e., Cmax and AUC increases were proportional to the dose increase although Cmax for elsiglutide increased less than expected on Day 4 at doses above 60 mg/day. The t1/2Z, CL/F and Vz/F were dose independent.

Study TIDE-11-10 showed that the pharmacokinetics of elsiglutide and its metabolites ZP2242 and ZP2712 varied considerably across patients and over multiple days.

Overall, PK data generated in the framework of Phase 1 and Phase 2a studies, indicated a rapid elimination of both the parent compound and its metabolites, which allow to predict a very modest, if any, accumulation at steady state.

Safety

Safety data in humans are derived from two phase I dose-escalation studies and one phase II study, which included 202 human subjects (36 healthy subjects and 166 colon and colorectal cancer patients receiving 5-FU-based chemotherapy), 117 of whom received active treatment.

In the first dose-escalation study (06-013) in healthy volunteers, the maximum tolerated dose (MTD; defined in this study as the dose level immediately prior to the dose level at which further dose escalation was halted due to the study ‘Stopping Rules’) was 9.6 mg i.v. and 3 mg s.c., as an AE that met the study stopping rules was observed in the 19.2 mg i.v. group (moderate positional lightheadedness) and in the 6 mg s.c. group (moderate systolic hypotension). Thirteen of 27 subjects included in this study experienced at least 1 treatment-emergent adverse event (TEAE) judged by the investigator as having either a possible, probable or definitive relation to the study medication. The most commonly reported related TEAEs being postural dizziness, nausea, and injection site erythema.

In the second dose-escalation study (TIDE-09-04) in cancer patients an independent data safety monitoring board supervised the dose escalation process. A dose of 93 mg/day administered for 4 consecutive days was reached and no dose limiting toxicities occurred, proving that elsiglutide was better tolerated than foreseen after study 06-013.

In studies TIDE-09-04 and TIDE-11-10 the overall pattern of TEAEs was as expected for patients with cancer receiving chemotherapy. In both studies the safety profiles of elsiglutide and placebo were generally similar. More patients in the elsiglutide group reported TEAEs compared to patients in the placebo group, mostly due to injection site reactions in the elsiglutide group. No serious or severe injection site reactions were observed.

In study TIDE-09-04, out of the 28 treated patients (21 on active treatment and 7 on placebo), 9 (32.1%; 7 patients in elsiglutide group and 2 patients in placebo group) experienced at least one TEAE judged by the investigator to be at least possibly related to treatment. In study TIDE-11-10, out of the 138 treated patients (69 on elsiglutide and 69 on placebo), 8 (11.6%), all in the elsiglutide group, had at least one TEAE judged by the investigator to be at least possibly related to study-drug. The most frequent related TEAEs overall in both studies were injection site events (in particular injection site erythema and injection site pain) followed by constipation.

In all three studies clinical laboratory, vital sign, ECG, and physical examination data did not reveal any clinically significant abnormal finding. No deaths or SAEs related to the study drug were reported.

Hence, on the basis of the human safety data collected so far, no safety concerns have been raised.

TIDE-11-10 Phase II Proof of Concept Study to Evaluate the Efficacy of Elsiglutide Administered as 24 mg Daily s.c. Bolus Injections for 4 Consecutive Days in Preventing CID in Patients with Colorectal Cancer Receiving 5-FU Based Chemotherapy (FOLFOX4 or FOLFIRI Regimen)

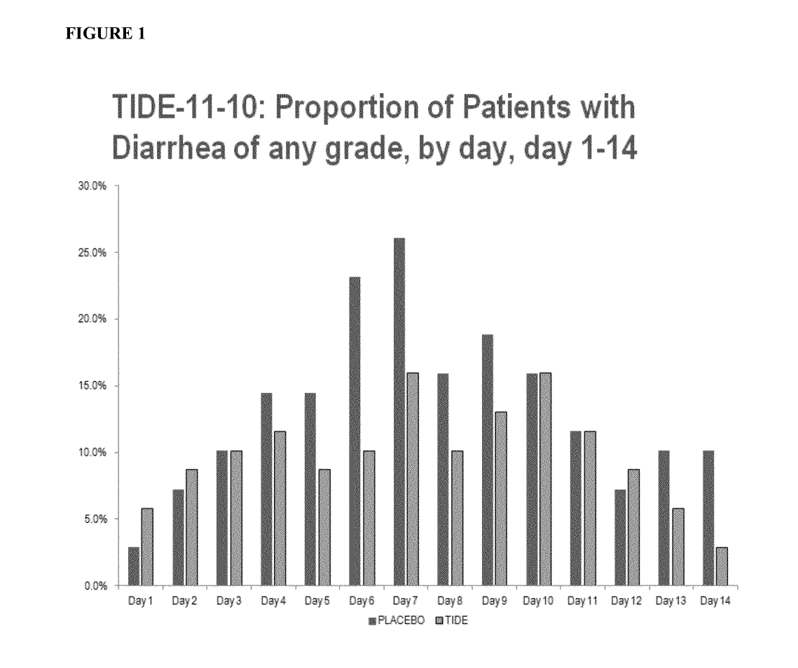

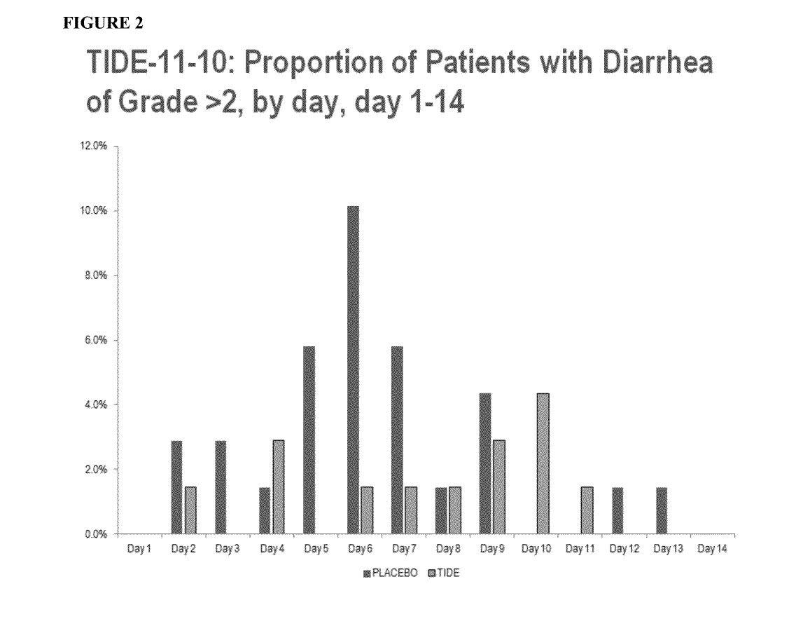

The formulation used in clinical trials is a lyophilized sterile powder for s.c. administration after reconstitution with sterile water for injection. The efficacy results obtained in study TIDE-11-10 indicate that elsiglutide had a preventive effect on the occurrence of grade ≧2 diarrhea (see FIGS. 1 and 2). This study also included an evaluation of the levels of citrulline, an amino acid mainly produced by enterocytes, a decrease of which is indicative of an intestinal mucosal damage following chemotherapy. Objectives: The main objective of TIDE-11-10 proof of concept study was to obtain data on the efficacy of elsiglutide in preventing CID in patients with colorectal cancer receiving 5-FU based chemotherapy (FOLFOX4 or FOLFIRI regimen) in comparison to placebo. In addition, safety and tolerability of the administered repeated doses of elsiglutide were evaluated, and the pharmacokinetics (PK) of elsiglutide and its metabolites ZP2242 and ZP2712 were investigated in a subset of patients in each treatment arm.

Methodology: This was a phase II, multicenter, double-blind, randomized, placebo-controlled, two-stage, proof of concept study with an interim futility analysis in colorectal cancer patients receiving 5-FU-based chemotherapy (FOLFOX4 or FOLFIRI) and administered elsiglutide subcutaneously (s.c.) for 4 consecutive days.

138 patients received a daily dose of 24 mg elsiglutide (or placebo practically identical in composition to the active study drug) via a single s.c. injection for 4 consecutive days, starting from the first day of chemotherapy administration. The patients were hospitalized at least until Day 3. Further visits were scheduled for Days 4, 5 and 15 and for a Follow-up Visit on Day 28-32. Safety and tolerability were monitored throughout the study.

Diagnosis and Main Criteria for Inclusion: Female and male patients of at least 18 years of age with confirmed diagnosis of colorectal cancer and an Eastern Cooperative Oncology

Group (ECOG) performance status ≦2, chemotherapy-naïve, and scheduled to receive a FOLFOX4 or FOLFIRI chemotherapy regimen.

ECOG PERFORMANCE STATUS*

| Grade | ECOG |
|---|---|
| 0 | Fully active, able to carry on all pre-disease performance without |
| | restriction |
| 1 | Restricted in physically strenuous activity but ambulatory and |
| | able to carry out work of a light or sedentary nature, e.g., light |
| | house work, office work |
| 2 | Ambulatory and capable of all selfcare but unable to carry out |
| | any work activities. Up and about more than 50% of waking |
| | hours |
| 3 | Capable of only limited selfcare, confined to bed or chair more |
| | than 50% of waking hours |
| 4 | Completely disabled. Cannot carry on any selfcare. Totally |
| | confined to bed or chair |
| 5 | Dead |

*As published in Oken et al., Toxicity And Response Criteria Of The Eastern Cooperative Oncology Group, Am J Clin Oncol 5: 649-655, 1982.

Efficacy: The endpoint of primary interest was:
Number of patients experiencing no diarrhea from Day 1 to Day 14

Secondary endpoints were:
Proportion of patients experiencing grades ≧2 diarrhea at each day from Day 1 to Day 14 according to National Cancer Institute Common Terminology Criteria for Adverse Events (NCI-CTCAE v. 4.03);
Worst grade of diarrhea according to NCI-CTCAE at each day from Day 1 to Day 14;
Time to occurrence of diarrhea, defined as the first day in which a grade ≧1 diarrhea was assessed (from Day 1 to day 14);
Number of days with presence of grade ≧1 diarrhea (from Day 1 to Day 14);
Number of days with presence of grade ≧2 diarrhea (from Day 1 to Day 14);
Number of days with presence of at least one bowel movement accompanied by urgency (from Day 1 to 14);
Number of days with presence of at least one episode of fecal incontinence (from Day 1 to 14);
Proportion of patients who required i.v. fluids due to CID (from Day 1 to 14);
Proportion of patients who required changes to the primary therapy (chemotherapy dose reduction, delay or change to regimen) due to CID as of Day 2, Day 14 and as of Day 28;
Proportion of patients who used rescue medication (i.e. medication used for treatment of diarrhea) from Day 1 to Day 14.

In addition, the proportion of patients who were limited concerning self-care Activities of Daily Living (ADL), the number of stools per day, the number of bowel movements accompanied by urgency per day, and the number of episodes of fecal incontinence per day were summarized for Day 1 to Day 14. Mean blood concentrations of citrulline (a biomarker for intestinal integrity, a decrease being indicative of an intestinal mucosal damage following chemotherapy) were summarized by treatment group for baseline, Day 5, and Day 15 including changes compared to baseline.

Summary—Conclusions:

For the overall trial, i.e. Stage 1 and Stage 2, (69 patients in each treatment group), superiority of elsiglutide to placebo was to be concluded if the difference in the number of responders (elsiglutide—placebo) was larger than or equal to 5. More patients were responders, i.e. had no diarrhea, in the elsiglutide group (43 patients) than in the placebo group (39 patients). While a trend toward superiority was shown, superiority of elsiglutide over placebo could not be statistically demonstrated.

Number of Responders¹ in the Period from
Day 1 to Day 14 Intent-to-treat Set

| | |
|---|---|
| Placebo | 24 mg/day Elsiglutide |
| N = 69 | N = 69 |

| | n | (%) | n | (%) |
|---|---|---|---|---|
| Responder | 39 | (56.5) | 43 | (62.3) |
| Non-responder | 30 | (43.5) | 26 | (37.7) |

¹Responder was defined as a patient experiencing no diarrhea
N = number of patients in treatment group, n = number of patients with data available, % = percentage based on N.

A higher frequency of diarrhea grade ≧2 was observed in the placebo group (15 patients) compared to the elsiglutide group (5 patients): the difference was more evident at Days 5, 6, and 7. See FIG. 2.

Grade of Diarrhea According to NCI-CTCAE version
4.03 (Day 1 to Day 14) Full Analysis Set

| | |
|---|---|
| Placebo | 24 mg/day Elsiglutide |
| N = 69 | N = 69 |

| Grade¹ | n | (%) | n | (%) |
|---|---|---|---|---|
| Grade 0 | 39 | (56.5) | 43 | (62.3) |
| Grade 1 | 15 | (21.7) | 21 | (30.4) |
| Grade 2 | 14 | (20.3) | 4 | (5.8) |
| Grade 3 | 1 | (1.4) | 1 | (1.4) |
| Grade <2 | 54 | (78.3) | 64 | (92.8) |
| Grade ≧2 | 15 | (21.7) | 5 | (7.2) |

¹Worst grade of diarrhea Day 1 to Day 14
NCI-CTCAE = National Cancer Institute Common Terminology Criteria for Adverse Events, % = percentage based on N.

In both treatment groups, the incidence of diarrhea increased on Day 2 and remained high up to Day 9. Diarrhea occurred most often 5 to 8 days after the first administration of chemotherapy.

Mean citrulline levels were similar in the treatment groups at baseline (placebo, 32.7 μmol/L; elsiglutide, 33.5 μmol/l). Mean levels decreased between Baseline and Day 5 in both treatment groups, with a less pronounced decrease in the elsiglutide group than in the placebo group. Between Day 5 and Day 15, mean citrulline levels increased in both treatment groups. In the elsiglutide group, mean citrulline levels at Day 15 were slightly increased compared to baseline (Day 15: placebo, 29.7 μmol/l; elsiglutide, 36.5 μmol/l).

### Example 2

### Follow-On Clinical Trial to Determine Optimal Dose and Regimen for Elsiglutide Effect on CID

TIDE-13-22: Randomized, Double Blind, Parallel Group, Placebo-Controlled, Dose Finding Study in Colorectal Cancer Patients Receiving 5-FU Based Chemotherapy to Assess the Efficacy of Different Doses of s.c. Elsiglutide in the Prevention of Chemotherapy Induced Diarrhea (CID)

The use of chemotherapy regimens for colorectal cancer treatment evolves continuously in all countries. In order to have a homogeneous patient population, the present study is planned to be conducted on patients with colorectal cancer scheduled to receive any FOLFOX (fluorouracil, folinic acid and oxaliplatin) or FOLFIRI (fluorouracil, folinic acid and irinotecan) chemotherapy regimens. These regimens have been described to cause CID in 30-80% of treated patients (Cherny, J Pain Symptom Manage 2008; 36:413-23; Arnold et al., J Support Oncol 2005; 3:227-232; Tournigand et al., J Clin Oncol. 2004; 22:229-237). Considering that monoclonal antibodies are often used together with standard chemotherapy regimens, this study also includes an additional patient population that is scheduled to receive FOLFOX or FOLFIRI together with monoclonal antibodies such as bevacizumab, cetuximab, panitumumab, or others, in order to gather preliminary results in this patient population. The present study aims at further investigating the efficacy of elsiglutide and identifying the most appropriate s.c. dosage. The dosages for the present study are 10 mg/day, 20 mg/day, and 40 mg/day each administered s.c. on 4 consecutive days.

Pharmacological preclinical studies in rats have shown that at a dose of 400 nmol/kg elsiglutide prevents 5-Fluorouracil (5-FU)-induced small intestinal atrophy and diarrhea, attenuates body weight loss and decreases mortality in rats. Elsiglutide also decreases the severity of irinotecan-induced diarrhea, including late-onset diarrhea, decreases body weight loss and lethality and enhances animal recovery. Moreover, elsiglutide has an intestinotrophic effect and histopathological observations show that it drastically reduces irinotecan induced severe intestinal damage. The dose of 400 nmol/kg corresponds to 1.7 mg/kg which in term of HED (human equivalent dose) results in approximately 20 mg (see guidance “Estimating the Maximum Safe Starting Dose in Initial Clinical Trials for Therapeutics in Adult Healthy Volunteers”, FDA July 2005). In addition PK/pharmacodynamic (PD) data in different animal species indicate that a dose of 20 mg/day could be considered the lowest efficacious dose.

The phase I and II studies described above (TIDE-09-04 and TIDE-11-10) have shown that the tolerability of the product is better than originally expected based on study 06-013. In study TIDE-09-04 a dose of 93 mg/day on 4 consecutive days was reached with no dose limiting toxicity. On the basis of pharmacological and clinical data (TIDE-09-04 and TIDE-11-10), it was deemed appropriate in the present study to consider a 4-day administration, starting from the day of chemotherapy. In study TIDE-11-10 a 24 mg/day dose administered s.c. on 4 consecutive days has shown some evidence of efficacy.

The present study aims at further investigating the efficacy of elsiglutide and identifying the most appropriate s.c. dosage. The dosages are 10 mg/day, 20 mg/day, and 40 mg/day each administered s.c. on 4 consecutive days.

A placebo treatment is included in this study as a control group in which the incidence of CID can be assessed. Moreover, besides clinical evaluation of the occurrence of diarrhea, the plasma level of citrulline, is also evaluated in order to establish its potential role as a biomarker of mucosal repair. Reduced citrulline levels are suggestive of intestinal failure (Crenn et al., Clin Nutr. 2008; 27(3):328-339) and have also been observed to be related with intestinal mucosal damage following chemotherapy (Herbers et al., Ann Oncol. 2010; 21(8):1706-1711).

### Study Objectives

The primary objective of the present study is to compare the efficacy of 3 s.c. doses of elsiglutide vs placebo and vs each other in the prevention of CID in colorectal cancer patients treated with 5-FU based chemotherapy (FOLFOX or FOLFIRI) with no addition of a monoclonal antibody.

As a secondary objective, the efficacy of 3 s.c. doses of elsiglutide vs placebo and vs each other in the prevention of CID in colorectal cancer patients treated with 5-FU based chemotherapy (FOLFOX or FOLFIRI) given in combination with a monoclonal antibody is explored.

### Investigational Plan

This is a randomized, stratified, double-blind, double-dummy, parallel group, placebo-controlled, dose finding, multicenter, multinational, phase II study in patients with colorectal cancer receiving 5-FU-based chemotherapy (FOLFOX or FOLFIRI) to assess the efficacy of different doses of s.c. elsiglutide in the prevention of CID. Chemotherapy naïve or non-naïve patients receive, starting from the day of chemotherapy administration, a single daily dose subcutaneously (s.c.) of elsiglutide 10, 20 or 40 mg or placebo for 4 consecutive days. Each patient is in the study for 3 consecutive chemotherapy cycles (14 days each). The treatment period for each patient is 4 consecutive days at each of the first two chemotherapy cycles.

The study includes 480 patients receiving 5-FU-based chemotherapy (FOLFOX or FOLFIRI) (“Target population”), and an additional group of up to 120 patients receiving the 5 FU-based chemotherapy in combination with a monoclonal antibody (“Additional population”).

TABLE 1
Study populations and treatment groups

| | 5-FU-based | 5-FU-based |
|---|---|---|
| | chemotherapy | chemotherapy + MAb |
| Treatment group: | Target population | Additional population |
| Placebo | 120 patients | Max 30 patients |
| 10 mg/day elsiglutide | 120 patients | Max 30 patients |
| 20 mg/day elsiglutide | 120 patients | Max 30 patients |
| 40 mg/day elsiglutide | 120 patients | Max 30 patients |
| Total per population | 480 patients | Max 120 patients |

| | |
|---|---|
| Total overall | Max 600 patients |

Within each population, patients are randomly allocated to receive one of 4 treatments (3 doses of elsiglutide or placebo) at Day 1, before the start of chemotherapy. The randomization is stratified by chemotherapy regimen (FOLFOX or FOLFIRI) and by country.

### Inclusion Criteria

1. Written informed consent;
2. Male or female patient >18 years of age;
3. Histologically or cytologically confirmed diagnosis of colorectal cancer;
4. Patients scheduled to receive at least 3 consecutive cycles of the same regimen of FOLFOX or FOLFIRI (Oxaliplatin/Irinotecan, Folinic acid, 5-FU).
5. Only for the Additional Population: Patient scheduled to receive monoclonal antibodies in combination with FOLFOX or FOLFIRI chemotherapy regimen;
6. A performance status of ≦2 according to the Eastern Cooperative Oncology Group (ECOG) scale;
7. Non-childbearing female patient or female patient of childbearing potential using reliable contraceptive measures and having negative pregnancy test before treatment administration;
8. Able to read, understand, follow the study procedure and complete patient diary.

### Efficacy Assessments

Patients preferably exhibit efficacy on the basis of one or more of the primary or secondary efficacy endpoints, as described below. The efficacy assessment is based both on data recorded by the patient and on the clinical assessment of the Investigator. Patients are asked to record on a daily basis in a e-Diary information related to her/his bowel movements (including time, number and consistency of stools (following the Bristol Stool Form Scale as described in Lewis and Heaton, Scand J Gastroenterol. 1997; 32 (9):920-924), urgency and fecal incontinence), limitations in activity of daily living (ADL), use of rescue medications due to diarrhea and abdominal discomfort. Moreover, patients are requested to report daily the occurrence of diarrhea. The patient e-Diary is to be filled in daily from Day 1 to Day 14 of Cycles 1, 2 and 3.

The occurrence of events of diarrhea over the 14-day period following each chemotherapy is assessed by the investigator based on the information collected in the patient's eDiary; severity of each event of diarrhea is graded by the Investigator according to NCI-CTCAE v.4.03 scale. The maximum grade assigned to any of the individual events is identified. A maximum Grade ≧2 diarrhea is considered for the primary endpoint.

In addition, the Investigator assigns a unique grade to the whole 14-day period (“overall grade”, based on NCI-CTCAE v.4.03 scale).

Plasma levels of citrulline are measured in all patients to evaluate its potential mucosa-protective effect. For this purpose, blood samples are taken at Screening, Day 2 of Cycle 1 (before study drug administration), Day 5 and Day 15 of the first 2 chemotherapy cycles, i.e. when the study drug is administered, and at Follow-up.

Safety is assessed based on adverse events (AEs), physical examinations, vital signs, clinical laboratory test results, immunogenicity data and 12-lead electrocardiograms (ECGs).

The PK of elsiglutide and its active metabolites ZP2242 and ZP2712 is assessed in all patients randomized to each of the study treatments and consenting to participate in PK sampling. The individual's dense plasma concentration-time data is evaluated and the standard PK parameters are estimated. The dose-proportionality is also investigated in the tested dose range between 10 and 40 mg. The influence of possible demographic and therapeutic covariates on the PK parameters and their variability is investigated by both a two-stage population PK approach and non-linear mixed effect modeling. The possible relationship between exposure to elsiglutide and its metabolites and efficacy measures is explored.

Patient's health related quality of life is measured by using the EQ-5D-3L questionnaire.

### (a) Primary Efficacy Endpoint

Proportion of patients experiencing a maximum Grade ≧2 diarrhea during the first cycle of chemotherapy (from Day 1 to Day 14 of Cycle 1) in Target population.

### (b) Secondary Efficacy Endpoints

Proportion of patients experiencing a maximum Grade ≧2 diarrhea during the first cycle of chemotherapy in the Additional population.

The following endpoints are considered for the Target population. They are also considered for the Additional population as relevant based on the number of patients available.
Proportion of patients experiencing a maximum Grade ≧2 diarrhea during the second and third cycle of chemotherapy;
Proportion of patients experiencing a maximum Grade ≧2 diarrhea over the first two cycles of chemotherapy (i.e. in at least one of the first two cycles);
Proportion of patients experiencing a maximum Grade 1, Grade 2, Grade 3, Grade 4, Grade 5 and any Grade (i.e. ≧1) diarrhea by cycle (Cycle 1, 2 and 3);
Proportion of patients experiencing an overall Grade ≧2 diarrhea by cycle (Cycle 1, Cycle 2 and Cycle 3);
Proportion of patients experiencing an overall Grade ≧2 diarrhea over the first two cycles of chemotherapy (i.e. in at least one of the first two cycles);
Proportion of patients experiencing an overall Grade 1, Grade 2, Grade 3, Grade 4, Grade 5 and any Grade (i.e. ≧1) by cycle (Cycle 1, 2 and 3);
Time to onset of first event of diarrhea of any Grade (i.e. ≧1) and time to onset of first event of diarrhea of Grade ≧2 (as assessed by the Investigator) by cycle (Cycle 1, 2 and 3);
Time to first day with diarrhea (as reported by patient in the eDiary) by cycle (Cycle 1, 2 and 3);
Cumulative duration (days) of any Grade (i.e. ≧1) diarrhea events and cumulative duration of Grade ≧2 diarrhea events (as assessed by the Investigator) by cycle (Cycle 1,2 and 3);
Cumulative duration (days) of diarrhea events (as assessed by the Investigator) by grade (Grade 1, Grade 2, Grade 3, Grade 4, Grade 5) and by cycle (Cycle 1, 2 and 3);
Number of events of diarrhea by grade (as assessed by the Investigator) by cycle (Cycle 1,2 and 3);
Number of days with presence of diarrhea (as reported by patient in the eDiary) by cycle (Cycle 1, 2 and 3);
Number of days with presence of at least one bowel movement with stools of consistency 6 or 7 (according to Bristol Stool Form Scale) accompanied by urgency or by fecal incontinence by cycle (Cycle 1, 2 and 3);
Number of days with presence of abdominal discomfort by cycle (Cycle 1, 2 and 3);
Number of days with limitation of self-care activities due to diarrhea by cycle (Cycle 1, 2 and 3);
Proportion of patients: who required i.v. fluids due to CID, who required changes to the primary therapy (chemotherapy dose reduction, delay or change to regimen) due to CID, who used rescue medication (i.e. medication used for treatment of diarrhea) by cycle (Cycle 1, 2 and 3);
Proportion of patients having a maximum Grade ≧2 diarrhea—shift from Cycle 1 to Cycle 2 and from Cycle 2 to Cycle 3;
Proportion of patients having a maximum Grade ≧1 diarrhea—shift from Cycle 1 to Cycle 2 and from Cycle 2 to Cycle 3;
Proportion of patients having an overall Grade ≧2 diarrhea—shift from Cycle 1 to Cycle 2 and from Cycle 2 to Cycle 3;
Proportion of patients having an overall Grade ≧1 diarrhea—shift from Cycle 1 to Cycle 2 and from Cycle 2 to Cycle 3;
Time trend of the citrulline plasma concentrations in Cycles 1, 2 and 3.

### (c) Evaluation of the Severity of Diarrhea

Severity of diarrhea is classified by the Investigator according to the NCI-CTCAE, Version 4.03 (June 2010) as described in 2 below:

TABLE 2
National Cancer Institute Common Toxicity
Criteria for Diarrhea (CTCAE v. 4.03)
Adverse event: Diarrhea

| Grade 1 | Grade 2 | Grade 3 | Grade 4 | Grade 5 |
|---|---|---|---|---|
| Increase | Increase | Increase of ≧7 | Life- | Death |
| of <4 stools/ | of 4-6 | stools/day over | threatening | |
| day over | stools/ | baseline; | consequences; | |
| baseline; | day over | incontinence; | urgent | |
| mild increase | baseline; | hospitalization | intervention | |
| in ostomy* | moderate | indicated; | indicated | |
| output | increase in | severe increase | | |
| compared to | ostomy* | in ostomy* | | |
| baseline | output | output compared | | |
| | compared to | to baseline; | | |
| | baseline | limiting self care | | |
| | | ADL# | | |

Definition: a disorder characterized by frequent and watery bowel movements.
A Semi-colon indicates ‘or’ within the description of a grade
*Patients with any type of ostomy were excluded from the present study
#Self care ADL refers to bathing, dressing and undressing, feeding self, using the toilet, taking medication, and not bedridden.

### (d) Quality of Life Assessment

Patient's health related quality of life is measured using the EQ-5D-3L questionnaire developed by the EuroQol Group. Patients complete the questionnaire EQ-5D-3L at Screening, Day 5 and Day 15 of Cycles 1, 2 and 3 (Follow-Up). The questionnaire is part of the e-Diary. The EQ-5D 3L has been designed as an international, standardized, generic instrument that describes health status in 5 dimensions. It generates 3 types of data for each patient:
1. a profile indicating the extent of problems across the 5 dimensions
2. a weighted health index based on general population values
3. a score on the self-rated “thermometer”, indicating the patient's own assessment of their health status

The main objective of this evaluation is to assess whether or not elsiglutide at different dosages vs. placebo is associated with a positive impact in patients' HRQL from baseline.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for preventing or reducing the occurrence or severity of grade 2 or higher diarrhea resulting from an anti-cancer chemotherapy in a subject in need thereof, which method comprises administering to the subject a therapeutically effective amount of elsiglutide in an elsiglutide regimen, wherein the elsiglutide regimen preferably comprises daily administration of elsiglutide for four consecutive days commencing at the start of the chemotherapy cycle.

2. A method of preventing or reducing gastrointestinal (GI) damage and/or dysfunction resulting from an anti-cancer chemotherapy in a subject in need thereof, which method comprises administering to the subject a therapeutically effective amount of elsiglutide in an elsiglutide regimen, wherein the elsiglutide regimen comprises daily administration of elsiglutide for a plurality of consecutive days preferably commencing at the start of the chemotherapy cycle and ending prior to the conclusion of the chemotherapy cycle.

3. The method of claim 1, wherein said chemotherapy comprises antibody therapy with or without small-molecule chemotherapy.

4. The method of claim 1, wherein said chemotherapy comprises bevacizumab, cetuximab or panitumumab antibody therapy with or without small-molecule chemotherapy.

5. The method of claim 1, wherein said elsiglutide regimen comprises daily administration of elsiglutide for 2 to 6 days, and said chemotherapy cycle is 8 to 24 days.

6. The method of claim 1, wherein said elsiglutide regimen comprises daily administration of elsiglutide for 4 days.

7. The method of claim 1, wherein the elsiglutide regimen prevents or reduces the occurrence of grade 2 or higher diarrhea resulting from an anti-cancer chemotherapy on days 5 and/or 6 of a chemotherapy cycle.

8. The method of claim 2, wherein the elsiglutide regimen reduces GI damage and/or dysfunction on days 5 and/or 6 of a chemotherapy cycle.

9. The method of claim 2, wherein the GI damage and/or dysfunction associated with the anti-cancer chemotherapy is gastrointestinal mucositis or chemotherapy-induced diarrhea (CID).

10. The method of claim 9, wherein the CID is Grade ≧2 diarrhea as determined by National Cancer Institute Common Toxicity Criteria for Diarrhea (CTCAE v.4.03).

11. The method of claim 1, wherein the elsiglutide is administered during at least the first four consecutive days from the beginning of the chemotherapy cycle.

12. The method of claim 1, wherein the elsiglutide is administered for two cycles of chemotherapy during the first four consecutive days from the beginning of each chemotherapy cycle.

13. The method of claim 1, wherein the chemotherapy cycle is up to 14 days long.

14. The method of claim 1, wherein the chemotherapy cycle is 14 days or longer.

15. The method of claim 1, wherein the therapeutically effective amount of the elsiglutide is about 10-40 mg/day.

16. The method of claim 1, wherein the therapeutically effective amount of elsiglutide is selected from about 10 mg/day, about 20 mg /day, and about 40 mg/day.

17. The method of claim 1, wherein the anti-cancer chemotherapy comprises administration of one or more compounds selected from the group consisting of antimetabolites, alkylating agents, anticancer antibiotics, microtubule-targeting agents, topoisomerase inhibitors, alkaloids, antibodies, pyrimidine analogs, purine analogs, folate antagonists, epidipodophyllotoxins, DNA damaging agents, antiplatelet agents, platinum coordination complexes, hormones, hormone analogs, aromatase inhibitors, anti-angiogenic compounds, growth factor inhibitors, angiotensin receptor blockers, nitric oxide donors, antisense oligonucleotides, cell cycle inhibitors, differentiation inducers, mTOR inhibitors, mitochondrial dysfunction inducers, chromatin disruptors.

18. The method of claim 1, wherein the anti-cancer chemotherapy comprises administration of one or more compounds selected from the group consisting of 5-fluorouracil (5-FU), floxuridine, capecitabine, gemcitabine, cytarabine, irinotecan, doxorubicin (adriamycin), amsacrine, camptothecin, daunorubicin, dactinomycin, eniposide, epirubicin, etoposide, idarubicin, mitoxantrone, topotecan, lapatinib, oxaliplatin, cisplatin, carboplatin, folinic acid, methothrexate, erlotinib, sorafenib, and lapatinib.

19. The method of claim 1, wherein the anti-cancer chemotherapy comprises administration of oxaliplatin or irinotecan.

20. The method of claim 1, wherein the anti-cancer chemotherapy comprises administration of oxaliplatin or irinotecan in combination with cetuximab, bevacizumab, and/or panitumumab.

21. The method of claim 1, wherein anti-cancer chemotherapeutic agent(s) is administered at least during the first two consecutive days from the beginning of each chemotherapy cycle.

22. The method of claim 1, wherein the anti-cancer chemotherapy is administered as FOLFOX or FOLFIRI chemotherapy regimen.

23. The method of claim 1, wherein the elsiglutide is administered subcutaneously (s.c.).

24. The method of claim 1, wherein the elsiglutide is administered intravenously or intraperitoneally.

25. The method of claim 1, wherein the subject is a human.

26. The method of claim 1, wherein the subject has a cancer with performance status of ≦2 according to the Eastern Cooperative Oncology Group (ECOG).

27. The method of claim 1, wherein the subject is chemotherapy-naïve prior to the start of the first chemotherapy cycle.

28. The method of claim 1, further comprising measuring blood levels of citrulline in said subject before and after the elsiglutide administration.

29. A method of preventing or reducing gastrointestinal (GI) damage and/or dysfunction resulting from an administration of an anti-cancer chemotherapeutic agent in a subject in need thereof, which method comprises administering to the subject a therapeutically effective amount of elsiglutide in an elsiglutide regimen, wherein the elsiglutide regimen comprises daily administration of elsiglutide for a plurality of consecutive days commencing before, during or after the administration of the anti-cancer chemotherapeutic agent.

30. The method of claim 29, wherein the administration of elsiglutide is commenced prior to the administration of the anti-cancer chemotherapeutic agent.

31. The method of claim 29, wherein the administration of elsiglutide is commenced during the administration of the anti-cancer chemotherapeutic agent.

32. The method of claim 29, wherein the administration of elsiglutide is commenced after the administration of the anti-cancer chemotherapeutic agent is completed.

33. A method for preventing or reducing GI damage by maintaining citrulline levels in a subject receiving chemotherapy, comprising administering to said subject a therapeutically effective amount of elsiglutide.

34. The method of claim 33, wherein said chemotherapy comprises a cycle of chemotherapy, and said therapeutically effective amount of elsiglutide comprises daily administration of elsiglutide for a plurality of consecutive days beginning at the initiation of the cycle of chemotherapy.


SEQUENCE LISTING (2 sequences)

SEQ ID NO: 1 - protein, 39 aa, Artificial
  Synthetic
HGEGSFSSELSTILDALAARDFIAWLIATKITDKKKKKK

SEQ ID NO: 2 - protein, 33 aa, Homo sapiens
HADGSFSDEMNTILDNLAARDFINWLIQTKITD